(19) United States
(12) Patent Application Publication
(10) Pub. No.: US 2016/0280716 A1
(43) Pub. Date: Sep. 29, 2016

(54) PHARMACEUTICAL SALT FORMS OF CEPHARANTHINE AND TETRANDRINE

(71) Applicant: Southwest Research Institute, San Antonio, TX (US)

(72) Inventors: William E. BAUTA, San Antonio, TX (US); Joseph A. McDonough, Helotes, TX (US); Mathew P.D. MAHINDARATNE, San Antonio, TX (US); Robert A. DAVEY, San Antonio, TX (US)

(21) Appl. No.: 15/078,782

(22) Filed: Mar. 23, 2016

Related U.S. Application Data
(60) Provisional application No. 62/136,851, filed on Mar. 23, 2015.

(51) Int. Cl. C07D 491/22

(52) U.S. Cl. CPC C07D 491/22

(57) ABSTRACT

The present disclosure relates to the preparation of pharmaceutically acceptable salt forms of the medicinal alkaloids Cepharanthine and Tetrandrine with improved solubility and physicochemical properties compared with the free base form of these alkaloids.

### CROSS REFERENCE TO RELATED APPLICATIONS

The present application claims the benefit of the filing date of U.S. Provisional Application Ser. No. 62/136,851, filed Mar. 23, 2015, the teachings of which are incorporated herein by reference.

### FIELD OF THE INVENTION

The present disclosure relates to the preparation of pharmaceutically acceptable salt forms of the medicinal alkaloids Cepharanthine and Tetrandrine with improved solubility and physicochemical properties compared with the free base form of these alkaloids. The disclosure includes the application of these salt forms to the treatment of diseases including viral infections, hypertension, cancer, neutropenia and, in a narrower embodiment, filovirus infections such as those resulting from Ebola and Marburg viruses and sub-types thereof.

### BACKGROUND

There are currently no effective therapies for the treatment of filovirus infections such as those caused by Ebolavirus. Recent work has revealed that medicinal alkaloids from members of the botanical family Stephania can effectively treat and prophylactically protect against infection by Ebolavirus based on in vitro and in vivo experimental results. These alkaloids have been used extensively in Asia for a variety of ailments ranging from hypertension to cancer for many years and have been dosed orally. However, the very limited solubility (≦1 mg/mL) of these alkaloids in their free base forms is an impediment to their development as oral therapeutics or via alternative routes of administration such as IV (intravascular administration), IP (intraperitoneal injection) or IM (intramuscular injection).

### BRIEF DESCRIPTION OF THE DRAWINGS

The above-mentioned and other features of this disclosure, and the manner of attaining them, will become more apparent and better understood by reference to the following description of embodiments described herein taken in conjunction with the accompanying drawings, wherein:

FIG. 1 illustrates the structure of Cepharanthine (CEPN);

FIG. 2 illustrates the structure of Tetrandrine (TETN);

FIG. 3 illustrates PXRD data for the Cepharanthine 2 HBr salt;

FIG. 3A illustrates the general structure of the Cepharanthine 2HBr salt.

FIG. 4 illustrates the Powder X-Ray Diffraction for Cepharanthine 2HCl salt;

FIG. 4A illustrates the general structure of the Cepharanthine 2HCl salt.

FIG. 5 illustrates the DSC thermogram for the Cepharanthine 2HCl salt;

FIG. 6 illustrates the Powder X-Ray Diffraction data for the Cepharanthine 2HCl salt recovered from ethyl acetate;

FIG. 7 illustrates the Powder X-Ray Diffraction data for the Cepharanthine 2HBr salt recovered from ethanol-water;

FIG. 8 illustrates the DSC thermograph for Cepharanthine 2HBr;

FIG. 9 illustrates the Powder X-Ray Diffraction data for Cepharanthine 2HBr recovered from ethyl acetate;

FIG. 10 illustrates the Powder X-Ray Diffraction data for Cepharanthine 2Pyruvic acid;

FIG. 11 illustrates the DSC thermograph for Cepharanthine 2Pyruvic acid;

FIG. 12 illustrates the Powder X-Ray Diffraction data for three (3) Cepharanthine 2HCl samples noted in Table 3;

FIG. 13 illustrates the DSC thermograph for sample 1 of Table 3;

FIG. 14 illustrates the DSC thermograph for sample 2 of Table 3; and

FIG. 15 illustrates the DSC thermograph of sample 3 of Table 3.

### SUMMARY

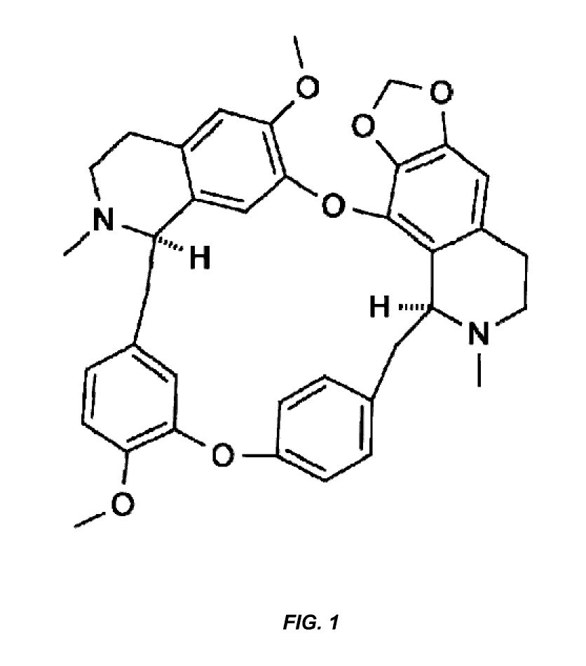

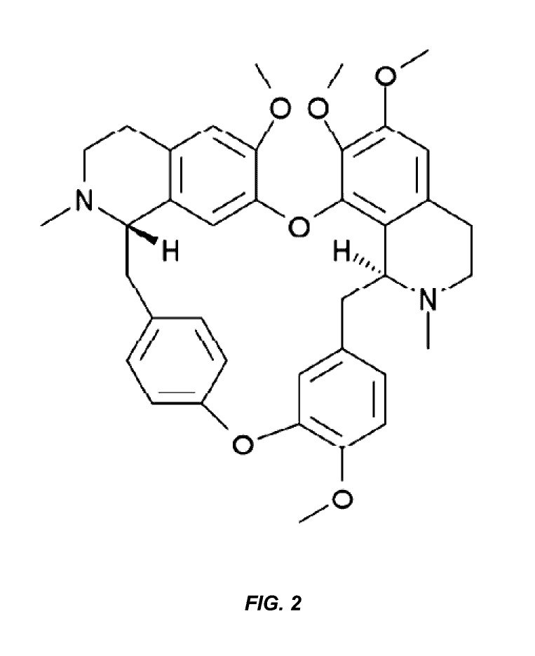

The present disclosure is directed at the preparation of salt forms for the medicinal alkaloids Cepharanthine (CEPN) and Tetrandrine (TETN). CEPN and TETN are illustrated, respectively, in FIGS. 1 and 2. Specifically the present disclosure is directed at the preparation of pharmaceutically acceptable salt forms that are isolated in particular polymorphic forms, which is a reference to the availability of the salt with distinguishing morphological structure.

### DETAILED DESCRIPTION

Pharmaceutically acceptable salt forms of CEPN and TETN, preferably of desired polymorphic forms, are prepared herein having superior solubility compared to the parent alkaloids and are applicable for the treatment of filovirus infection. It is contemplated herein that the pharmaceutically acceptable salts of the desired polymorphic forms will include various salts that may be sourced from either inorganic or organic type acids. Table 1 below identifies the contemplated inorganic and organic acids that may be employed.

TABLE 1
Pharmaceutically Acceptable Salt Forms of CEPN and TETN

| | |
|---|---|
| Inorganic | |
| acids | Organic Acids |

| | | | |
|---|---|---|---|
| Hydrochloric | Formic | Maleic | Benzoic |
| Hydrobromic | Acetic | Malonic | Glucoronic |
| Phosphoric | Methanesulfonic | Glutamic | Fumaric |
| Sulfuric | Tosic | Aspartic | |
| Nitric | Tartaric | Pyruvic | |
| | Lactic | Mucic | |

The pharmaceutically acceptable salt forms of the desired polymorphic form were prepared by adding a suitable acid from the list in Table 1 to a solution of an alkaloid and isolation of the resultant salt either by crystallization or solvent evaporation protocol to promote formation of the desired polymorph. In preferred embodiments the salt form may include either the mono- or the bis-salt and this may be further purified by recrystallization from a suitable solvent.

A representative sample preparation procedure follows with respect to CEPN. Both tertiary amine groups in the alkaloid CEPN are contacted with the selected acid in an appropriate solvent system. All reactions were carried out in ambient temperature and pressure. The acids selected for the final salt formulations were etheric HCl and liquid methanesulfonic (MSA) and pyruvic acids, all of which were monobasic. Thus, exact Cepharanthine:acid molar ratio of 1:2 was used in all preparations. Ethanol was preferred as the common solvent in all preparations, except for that of MSA salt to avoid the known neurotoxic ester formation of MSA with alcohols. In that instance, dichloromethane (DCM) was used as the alternative solvent since Cepharanthine is well soluble in DCM.

The process for the preparation of pyruvic acid salt was accomplished as follows. Pure Cepharanthine (1.82 g, 3.0 mmol) was completely dissolved in ethanol (25 mL) and 2 equivalents of pyruvic acid (0.53 g, 6 mmol) in ethanol was added slowly while stirring. Additional ethanol was used to complete the pyruvic acid transfer. The mixture was stirred further 30 min before ethanol was evaporated in reduced pressure. The gummy residue was dissolved again in dichloromethane (DCM) and evaporated to obtain a foamy solid. Thus, the purification was completed by precipitation using DCM/heptanes solvent system followed by filtration of the solid, washing the solid with a mixture of solvent systems containing 2-propanol, DCM, and heptanes, and drying the solid under vacuum to obtain partially shiny solid (2.14 g, 91% yield). Comparison of ¹H NMR spectra of both salt and Cepharanthine confirmed the formation of pyruvic acid salt.

The solubility of the salts prepared herein are identified below in Table 2.

TABLE 2
Solubility of Cepharanthine Salts in Water

| | | | Final Concentration |
|---|---|---|---|
| Sample ID | Description | Dilution needed | (mg/mL) |
| 1 | HCl salt | 1000x | 51.6 |
| 2 | HCl salt (diluted) | 1000x | 21.1 |
| 3 | HBr salt | 1000x | 17.1 |
| 4 | Benzoic Acid salt | 100x | 7.78 |
| 5 | Lactic Acid salt | 1000x | 41.2 |
| 6 | MSA salt | 20,000x | 800 |
| 7 | Pyruvic acid salt | 10,000x | 488 |

As can be seen from the above, preparation of pharmaceutically acceptable salts according to a preparation protocol that is contemplated to produce a particular resulting polymorphic salt structure is such that remarkably improved solubility is achieved which will then translate into more efficient drug delivery for treatment of filovirus infections. Such polymorphic structure may also be readily confirmed by other analytical techniques, including but not limited to x-ray diffraction and identification of relative intensity counts and/or scanning electron micrographs which identifies variables such as the aspect ratio or range of aspect ratios that may be present.

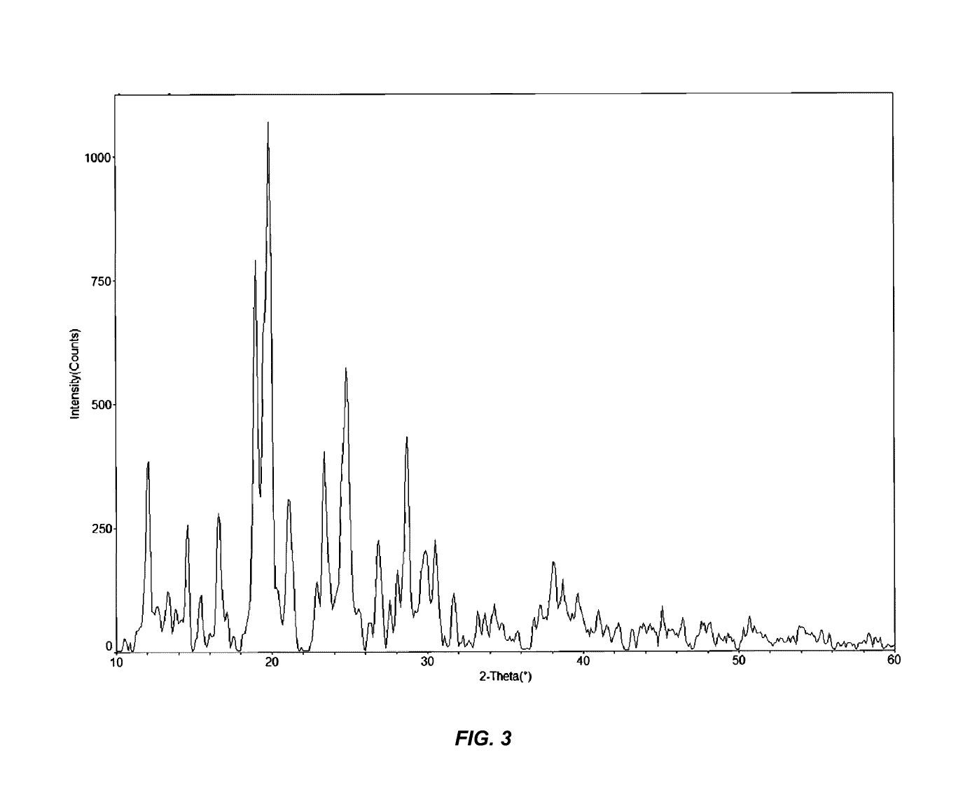

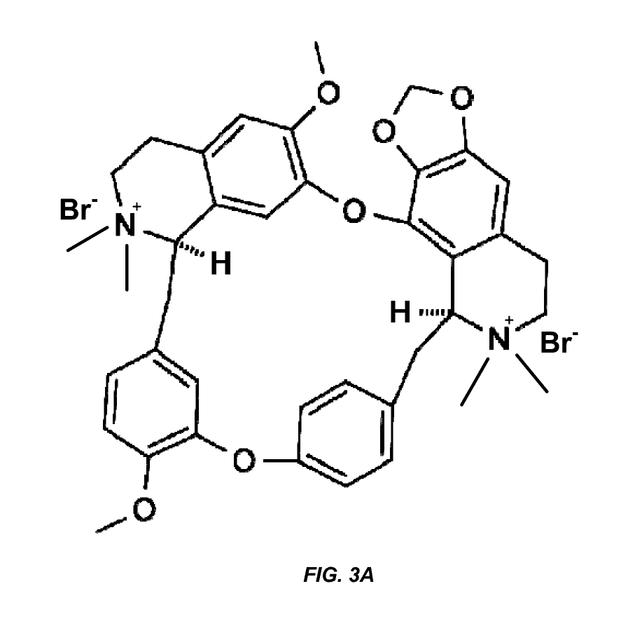

The process for the preparation of salt derivatives of Cepharanthine using various organic and inorganic acids can proceed as follows: the process comprises contacting both tertiary amine groups in the alkaloid Cepharanthine with the selected acid in an appropriate solvent system. All reactions were carried out in ambient temperature and pressure. The inorganic acids selected for the final salt formulations were HCl and HBr, which were obtained in either case as 2M solutions. The inorganic acids selected were methanesulfonic acid (MSA) and pyruvic acid, both of which were liquids. Since all of these acids were monobasic, the Cepharanthine to acid mole ratio of 1:2 was used in all salt preparations. Ethanol was preferred as the common solvent in all preparations, except for that of MSA salt to avoid the known neurotoxic ester formation of MSA with alcohols. In that instance, dichloromethane (DCM) was used as the alternative solvent since Cepharanthine is well soluble in DCM. FIG. 3 illustrates Powder X-Ray Diffraction (PXRD) data for the Cepharanthine 2HBr salt recovered from isopropanol. As can be seen, the PXRD indicates that a plurality of distinguishing x-ray diffraction peaks at 2 Theta angles of 10-35 degrees as compared to non-distinguishing x-ray diffraction peaks at 2 Theta angles of greater than 35 degrees, wherein said distinguishing x-ray diffraction peaks have relative intensity counts between 250-1250 at 2 Theta angles of 10-35 degrees and non-distinguishing x-ray diffraction peaks have relative intensity counts of less than 250 at 2 Theta angles of greater than 35 degrees. FIG. 3A illustrates the general structure of the Cepharanthine 2HCl salt.

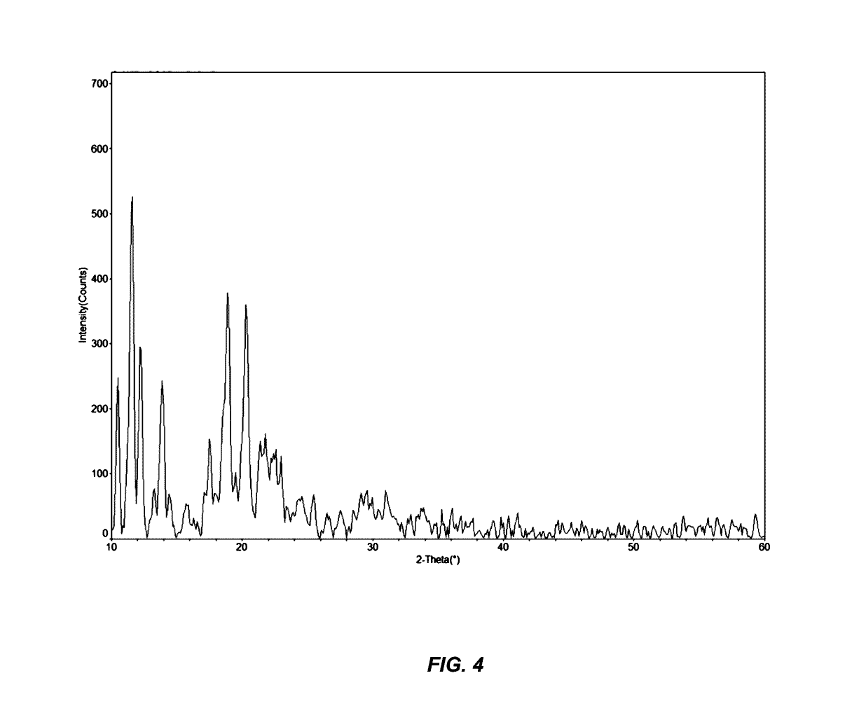

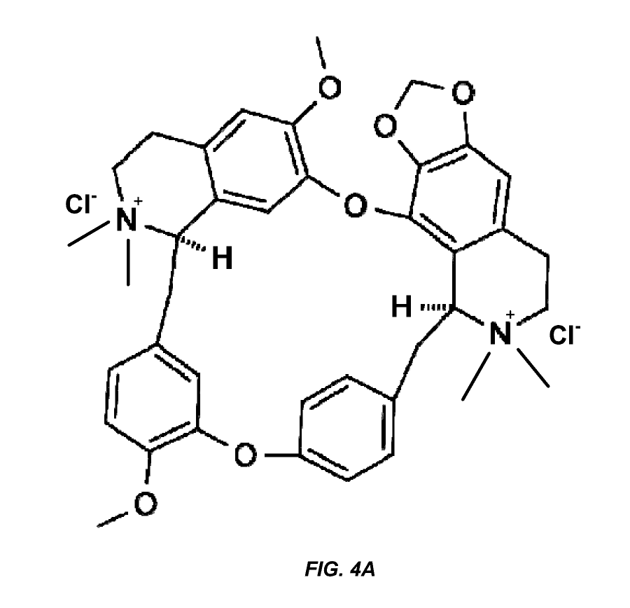

FIG. 4 illustrates the Powder X-Ray Diffraction (PXRD) for the Cepharanthine 2HCl salt. FIG. 4A illustrates the general structure of such salt. As can be seen, the PXRD indicates that a plurality of distinguishing x-ray diffraction peaks at 2 Theta angles of 10-25 degrees as compared to non-distinguishing x-ray diffraction peaks at 2 Theta angles of greater than 25 degrees, wherein said distinguishing x-ray diffraction peaks have relative intensity counts between 200-550 at 2 Theta angles of 10-25 degrees and non-distinguishing x-ray diffraction peaks have relative intensity counts of less than 100 at 2 Theta angles of greater than 35 degrees.

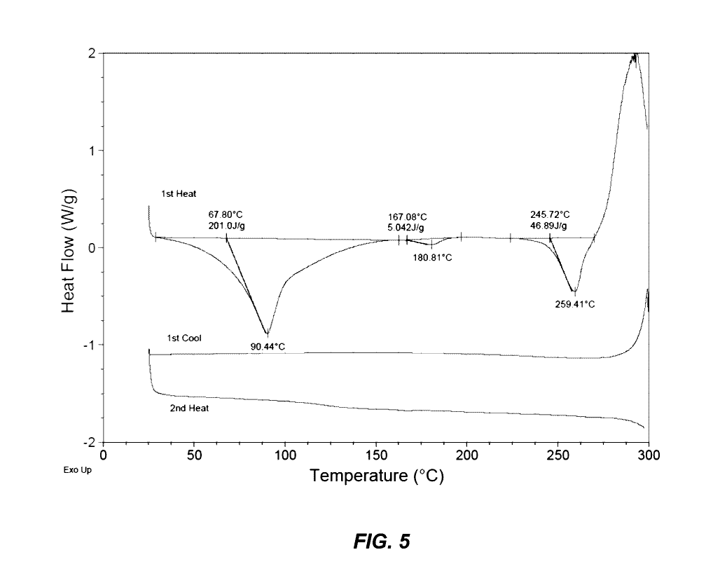

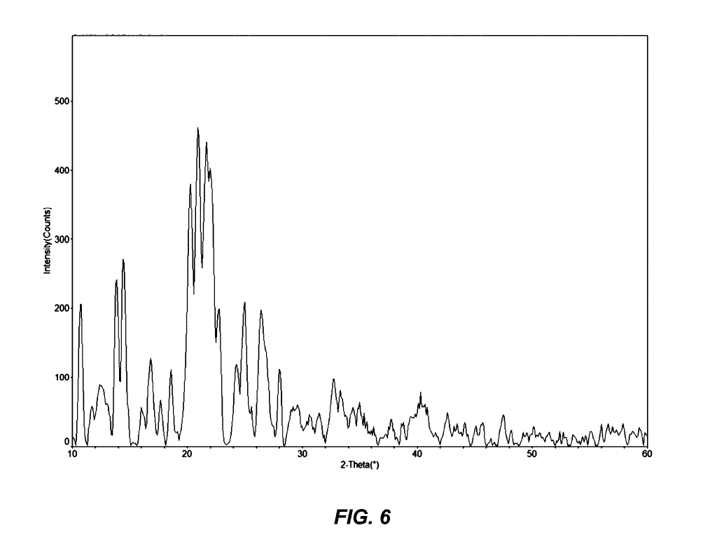

FIG. 5 illustrates the DSC thermogram for the Cepharanthine 2HCl salt. As can be seen, it identifies at least two peaks in the melting endotherms at temperatures in the range of 85° C. to 95° C. (90.44° C. peak identified) and in the range of 255° C. to 265° C. (259.41° C. peak identified). A relatively small endotherm peak is also observed in the range of 175° C. to 185° C. (180.81° C. peak identified). FIG. 6 illustrates the Powder X-Ray Diffraction (PXRD) data for Cepharanthine 2HCl salt recovered from ethyl acetate. As can be seen, the PXRD indicates that a plurality of distinguishing x-ray diffraction peaks at 2 Theta angles of 10-30 degrees as compared to non-distinguishing x-ray diffraction peaks at 2 Theta angles of greater than 30 degrees, wherein said distinguishing x-ray diffraction peaks have relative intensity counts between 150-500 at 2 Theta angles of 10-30 degrees and non-distinguishing x-ray diffraction peaks have relative intensity counts of less than 150 at 2 Theta angles of greater than 30 degrees.

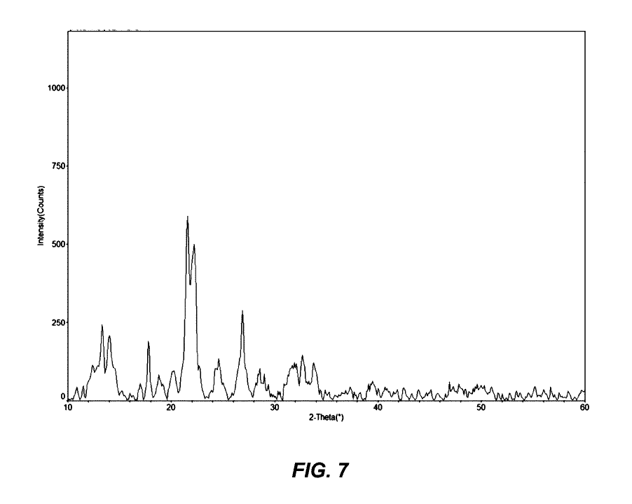

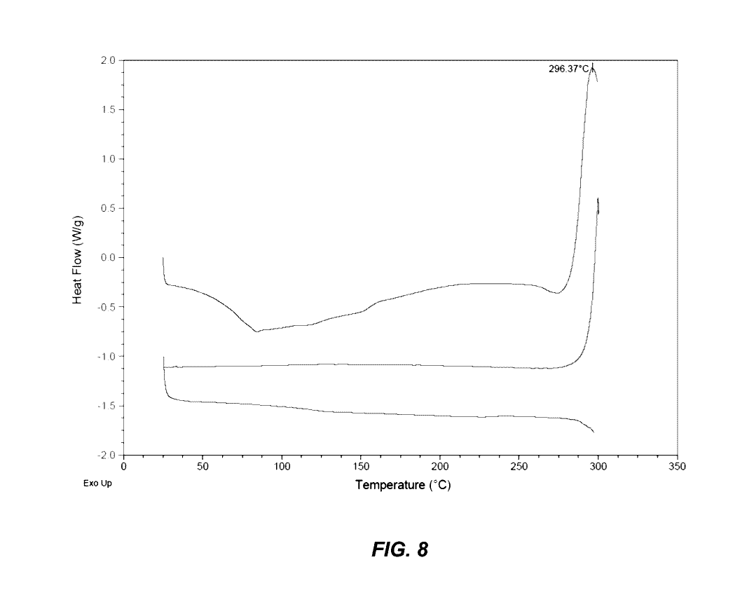

FIG. 7 illustrates the Powder X-Ray Diffraction (PXRD) data for the Cepharanthine 2HBr salt recovered from ethanol-water. As can be seen, the PXRD indicates that a plurality of distinguishing x-ray diffraction peaks at 2 Theta angles of 10-30 degrees as compared to non-distinguishing x-ray diffraction peaks at 2 Theta angles of greater than 30 degrees, wherein said distinguishing x-ray diffraction peaks have relative intensity counts between 200-550 at 2 Theta angles of 10-30 degrees and non-distinguishing x-ray diffraction peaks have relative intensity counts of less than 200 at 2 Theta angles of greater than 30 degrees. FIG. 8 illustrates the DSC thermograph for Cepharanthine 2HBr.

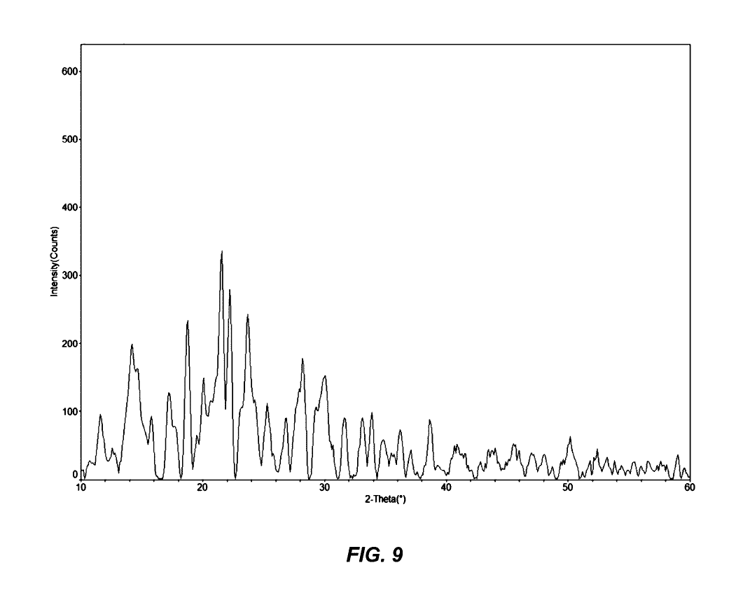

FIG. 9 illustrates the Powder X-Ray Diffraction (PXRD) data for Cepharanthine 2HBr recovered from ethyl acetate. As can be seen, the PXRD indicates that a plurality of distinguishing x-ray diffraction peaks at 2 Theta angles of 10-35 degrees as compared to non-distinguishing x-ray diffraction peaks at 2 Theta angles of greater than 35 degrees, wherein said distinguishing x-ray diffraction peaks have relative intensity counts between 150-400 at 2 Theta angles of 10-35 degrees and non-distinguishing x-ray diffraction peaks have relative intensity counts of less than 150 at 2 Theta angles of greater than 35 degrees.

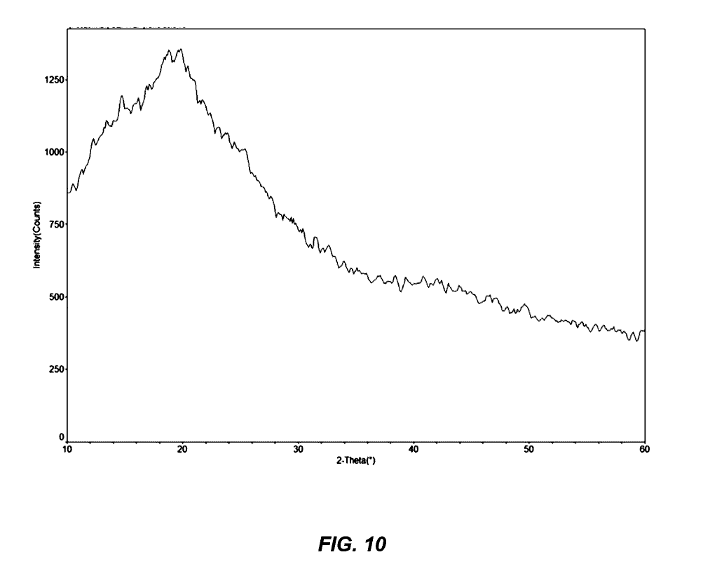

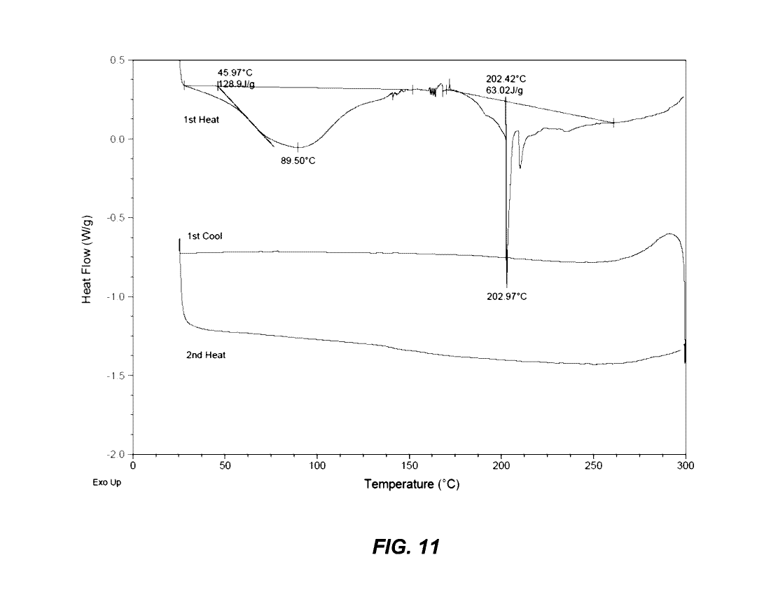

FIG. 10 illustrates the Powder X-Ray Diffraction (PXRD) data for Cepharanthine 2Pyruvic acid. FIG. 11 illustrates the DSC thermograph for Cepharanthine 2Pyruvic acid.

Table 3 below provides the data of three additional samples of Cepharanthine 2HCl recrystallized from ethanol-water.

TABLE 3
Data for CEPN 2HCl Salts Recrystallized from EtOH—H₂O

| Sample ID | CEPH 2HCl (g) | Recovery (g) | Recovery (%) |
|---|---|---|---|
| 1 | 1.0074 | 0.1739 | 17.2 |
| 2 | 1.0077 | 0.4367 | 43.3 |
| 3 | 1.0065 | 0.3651 | 36.3 |

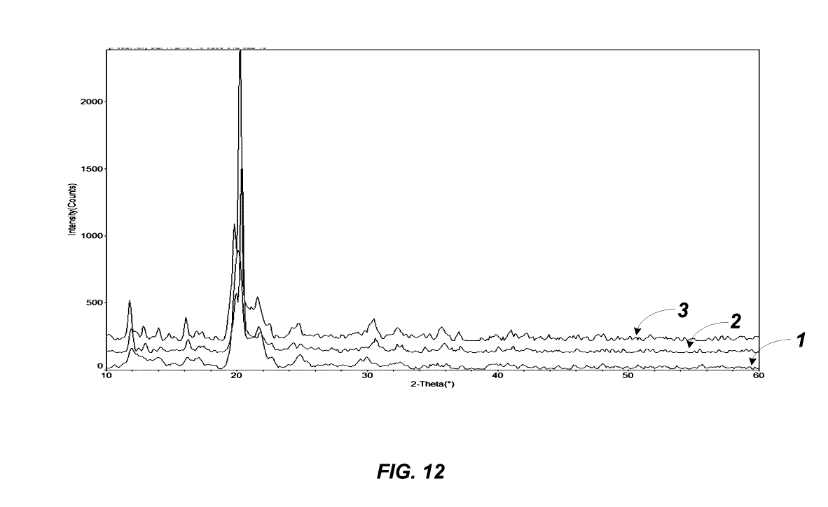

FIG. 12 provides the Powder X-Ray Diffraction (PXRD) data for the three (3) Cepharanthine 2HCl samples noted above in Table 3. As can be seen, the PXRD indicates that a plurality of distinguishing x-ray diffraction peaks at 2 Theta angles of 10-25 degrees as compared to non-distinguishing x-ray diffraction peaks at 2 Theta angles of greater than 25 degrees, wherein said distinguishing x-ray diffraction peaks have relative intensity counts between 500-2500 at 2 Theta angles of 10-25 degrees and non-distinguishing x-ray diffraction peaks have relative intensity counts of less than 500 at 2 Theta angles of greater than 25 degrees.

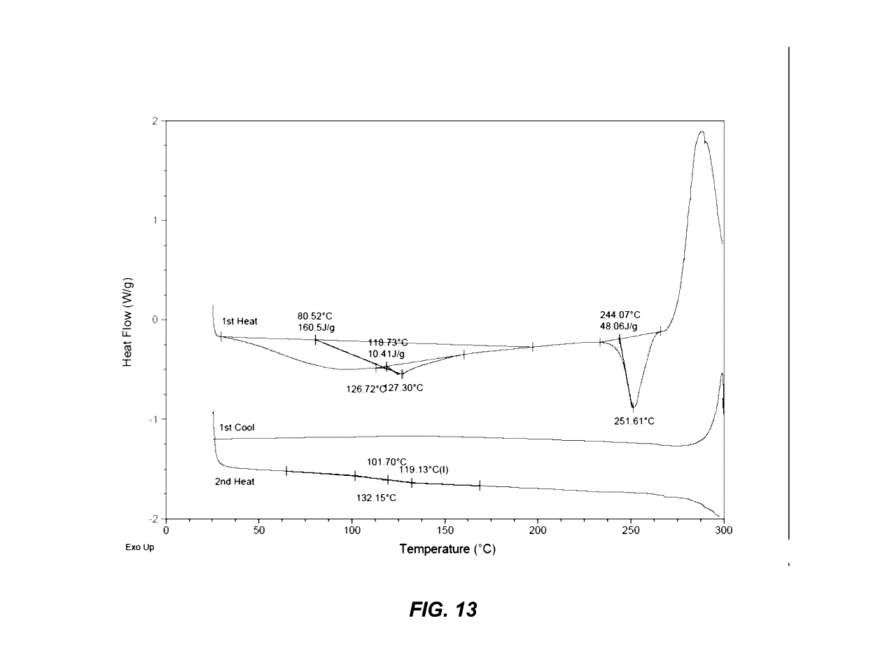

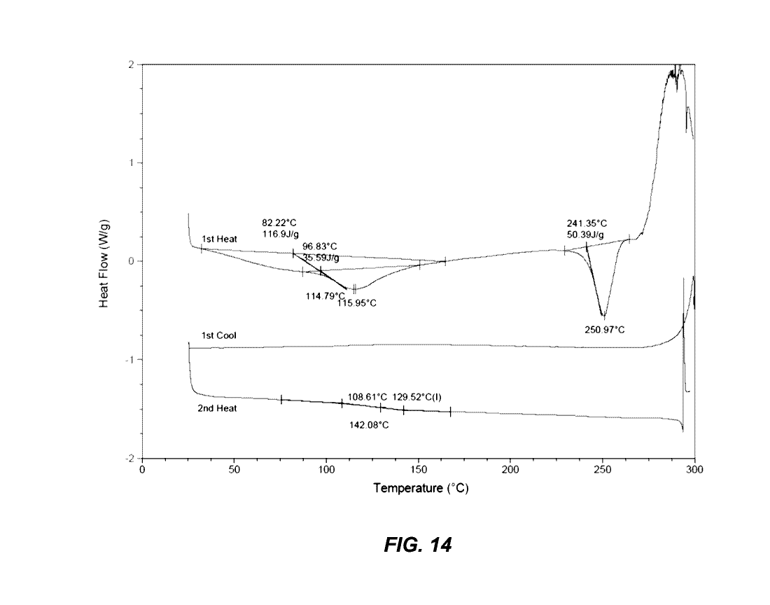

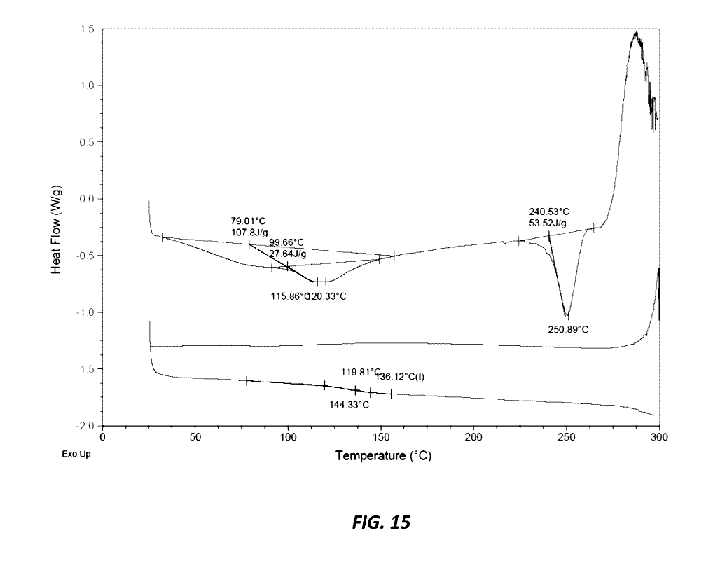

FIG. 13 provides the DSC thermograph of sample 1 from Table 3. FIG. 14 provides the DSC thermograph of sample 2 from Table 3. FIG. 15 provides the DSC thermograph of sample 3 from Table 3.

In one example, Cepharanthine bis-hydrobromide was obtained through the following preferred procedure. To a stirred solution of Cepharanthine (2.2 g, 3.626 mmol) in EtOH (40 mL) was added 2N hydrobromic acid (3.63 mL, 7.252 mmol). The mixture was stirred for 30 minutes. Diethyl-ether was added and the resulting slurry was filtered. The solid was recrystallized from 2-propanol and hexanes to obtain crystalline Cepharanthine bis-hydrobromide (2.27 g, 82%).

In the case of organic acid salts, briefly, the process for the preparation of pyruvic acid salt was accomplished as follows: pure Cepharanthine (1.82 g, 3.0 mmol) was completely dissolved in ethanol (25 mL) and 2 equivalents of pyruvic acid (0.53 g, 6 mmol) in ethanol was added slowly while stirring. Additional ethanol was used to complete the pyruvic acid transfer. The mixture was stirred further 30 min before ethanol was evaporated in reduced pressure. The gummy residue was dissolved again in DCM and evaporated to obtain a foamy solid. The solid was suspended in methyl tert-butyl ether (MTB), MTB supernatant was decanted off to remove excess pyruvic acid if present and remaining solvent was removed with vacuum. Attempts to crystallize the salt using 2-propanol, ethanol, and DCM were unsuccessful. Thus, the purification was completed by precipitation using a DCM/heptane solvent system followed by filtration of the solid, washing the solid with a mixture of solvent systems containing 2-propanol, DCM, and heptanes, and drying the solid under high-vacuum to obtain partially shiny solid (2.14 g, 91% yield). Comparison of H¹ NMR spectra of both salt and Cepharanthine (solvent: CDCl3) confirmed the formation of the pyruvic acid salt.

In yet another preferred procedure, Cepharanthine bis-pyruvate was formulated by adding a solution of pyruvic acid (0.528 g, 6.00 mmol) in EtOH (10 ml) to a stirred solution of Cepharanthine (1.82 g, 3.000 mmol) in EtOH (25 mL). The residual pyruvic acid was rinsed with EtOH (5 mL) and added to the Cepharanthine solution. The mixture was stirred for 1 hour and evaporated. The gummy solid was dissolved in dichloromethane and concentrated to afford a solid foam of Cepharanthine bis-pyruvate salt (2.20 g).

Cepharanthine bis-pyruvate was also preferably formulated by slow adding a solution pyruvic acid (27.6 g, 313.200 mmol) in dichloromethane (DCM) (40 mL) to a stirred solution of Cepharanthine (95.0 g, 156.60 mmol) in dichloromethane (DCM) (400 mL). The residual pyruvic acid was rinsed with DCM (50 mL) and added to the Cepharanthine solution. The mixture was stirred for 1 hour and concentrated to afford a light pink solid of Cepharanthine bis-pyruvate salt (122.1 g).

The process for the preparation of salt derivatives of Tetrandrine using various organic and inorganic acids can preferably proceed as follows: the process comprises contacting both tertiary amine groups in the alkaloid Tetrandrine with the selected acid in an appropriate solvent system. All reactions were preferably carried out in ambient temperature and pressure unless noted otherwise. The preferred inorganic acids selected for the final salt formulations were HCl and HBr, which were obtained in either case as 2M solutions. The organic acids selected were preferably methanesulfonic acid (MSA) and pyruvic acid, both of which were liquids. Ethanol was preferred as the common solvent in all preparations, except for that of MSA salt to avoid the known neurotoxic ester formation of MSA with alcohols.

A representative procedure with respect to inorganic acids follows with regard to Tetrandrine bis-hydrobromide (TETN-2HBr). Hydrobromic acid in 48% aq (1.10 ml, 9.67 mmol) is added to a stirred solution of TETN (3.01 g, 4.83 mmol) in DCM 22 (ml) and EtOH (192 ml). The reaction mixture was sealed and stirred at room temperature for 30 minutes and concentrated. The foam was placed on high vacuum overnight to afford yellow solid foam (3.80 g, 100% yield). The solid TETN-2HBr was suspended in and precipitated from various solvents, i.e., isopropyl alcohol (IPA), acrylonitrile (ACN), dichloromethane-methanol (DCM-MeOH), and methanol (MeOH), as set forth in Table 4, below. The crystallization products are also discussed in Table 4 below.

TABLE 4
TETN-2HBr Crystallization Conditions and Data

| | Crystallization Conditions | | |
|---|---|---|---|
| Crystallization | (mg of TETN-2HBr | Material after | |
| Solvent | in ml solvent) | Crystallization | Micrographs |
| Isopropyl | 142 mg in 13 ml | Yellow Needles | Crystalline |
| Alcohol | | | |
| Acrylonitrile | 20 mg in 5 ml heated | Light Yellow Solid | Glass |
| Dichloromethane- | 270 mg in (3 ml:1 ml) | | Glass/Crystalline |
| Methanol | DCM:MeOH, respectively | | |
| Methanol | 28.2 mg in 0.5 ml, heated to | White Solid | NA |
| | dissolve and slow evaporation | | |

A representative process for the preparation of Tetrandrine bis-phosphate (TETN-2H₂PO₄) is discussed further herein. Phosphoric acid in 85% aq. (0.53 ml, 7.83 mmol) was added to a stirred solution of Tetrandrine (2.44 g, 3.92 mmol) in dichloromethane (DCM) (40 ml) and ethyl alcohol (EtOH) (150 ml). White precipitate immediately formed. The reaction mixture was sealed and stirred at room temperature for 40 minutes and concentrated. The solid was placed on high vacuum overnight to afford white powder solid (3.22 g, 100% yield). The process was repeated for other solvents, i.e., isopropyl alcohol (IPA), dichloromethane-methanol (DCM-MeOH), methanol (MeOH), ethanol-methanol (EtOH-MeOH) solvent systems, as outlined in Table 5 below.

TABLE 5
TETN-2H₂PO₄ Crystallization Conditions and Data

| | Crystallization Conditions | | |
|---|---|---|---|
| Crystallization | (mg TETN-2H₂PO₄ in ml | Material after | |
| Solvent | solvent) | Crystallization | Micrographs |
| IPA | 100 mg in 20 ml, heated (1.5 | White Solid | Crystalline |
| | week slow evaporation) | | |
| DCM-MeOH | 400 mg in 50 ml (25 ml:25 ml) | Light Pink Solid | Crystalline + Glass |
| | DCM:MeOH, respectively, | | |
| | heated (slow evaporation) | | |
| MeOH | 125 mg in 10 ml MeOH, | Light Pink Solid | Crystalline + Glass |
| | heated (slow evaporation) | | |
| EtOH—MeOH | 26 mg in 7 ml (5 ml:2 ml) | White Solid | Glass |
| | EtOH:MeOH, respectively, | | |
| | (slow evaporation) | | |

Tetrandrine bis-hydrochloride was prepared according to the following representative procedure. To a stirred solution of Tetrandrine (2.49 g, 4.000 mmol) in ethanol (EtOH) (25 mL) was added 2N hydrochloric acid in diethyl ether (4.40 mL, 8.800 mmol) slowly resulting in a light brown precipitate. The precipitation was completed by adding methyl tert-butyl ether (MTB). The slurry was filtered and washed with 2 times with MTB. The solid was allowed to air dry to afford Tetrandrine bis-hydrochloride salt (3.30 g, 119%).

A further representative process to provide Tetrandrine bis-sulfate was prepared as follows. To a stirred solution of Tetrandrine (3.0 g, 4.817 mmol) in ethanol-dichloromethane (EtOH-DCM) (100 mL/20 mL) was added dropwise 18M sulfuric acid (0.535 mL, 9.634 mmol). The mixture was stirred overnight. The resulting white slurry was filtered to afford Tetrandrine bis-sulfate (3.28 g). Next, 640 mg of the solid was slurried with DCM (4 mL) and dissolved by adding MeOH (0.4 mL). The solution was slowly stirred and allowed to evaporate overnight resulting in white solid. A micrograph was obtained of the solid.

In the case of organic acids, a representative process for the preparation of Tetrandrine bis-methanesulfonate (TETN-2MSA) is discussed further herein. To a stirred solution of Tetrandrine (2.50 g, 4.01 mmol) in dichloromethane (DCM) (40 ml) was added methane sulfonic acid (MSA) (0.52 ml, 8.03 mmol). The reaction mixture was sealed and stirred at room temperature for 60 minutes and concentrated. The solid foam was placed on high vacuum overnight to afford yellow solid foam (3.20 g, 100% yield). The TETN-2MSA was crystallized by adding 1:1 ethanol (EtOH) and acetone (3 ml each) to a 40 ml vial of TETN-2MSA, which was then heated until the TETN-2MSA dissolved. The solution was then allowed to slowly evaporate in a fume hood for better air circulation to obtain white solid powder. Micrograph confirmed this provided a crystalline salt.

In another embodiment, the TETN-2MSA was dissolved in ethanol (EtOH). Hexane was added as an anti-solvent to provide a white precipitate. The mixture was allowed to slowly evaporate while cap off and letting air circulate in a fume hood to obtain a white powder. Micrograph analysis indicated the formation of crystals.

Table 6 outlines conditions for crystallization of the TETN-2MSA using ethanol-acetone, methanol, ethanol-hexane, water and ethanol.

TABLE 6
Crystallization Conditions and Data

| Crystallization | Crystallization Conditions | Material after | |
|---|---|---|---|
| solvent | (mg of TETN-2MSA in ml of solvent) | Crystallization | Micrographs |
| EtOH-Acetone | 310 mg in 6 ml EtOH:acetone (3 mL:3 mL), | White solid | Crystalline |
| | dissolved with heat and slow | powder | |
| | evaporation | | |
| MeOH | Heated to dissolve then slow evaporation | Clear oil | NA |
| EtOH-Hexane | Dissolved in EtOH and added hexane as a | White solid | Crystalline |
| | antisolvent to get ppt, slow evaporation | powder | |
| H₂O | 84 mg dissolved in 0.5 mL H₂O with slow | Oil/gummy | NA |
| | evaporation | | |
| EtOH | Dissolved in EtOH heated to dissolve, slow | White solid | NA |
| | evaporation | powder | |

Powder X-ray Diffraction (PXRD) analysis indicates that triclinic crystals were obtained for the ethanol-acetone process conditions of Table 6 above.

A representative process for the preparation of Tetrandrine bis-lactic acid (TETN-2LA) is discussed further herein. Lactic acid (0.65 ml, 8.70 mmol) was added to a stirred solution of Tetrandrine (2.70 g, 4.35 mmol) in dichloromethane (40 ml). The reaction mixture was sealed and stirred at room temperature for 60 minutes and concentrated. The solid foam was placed on high vacuum overnight to afford white solid foam (3.50 g, 100% yield).

Table 7, below, provides crystallization conditions and observations using water, acetone-hexane, methanol (MeOH), isopropyl alcohol (IPA), ethanol-hexane (EtOH-hexane) and ethanol (EtOH) solvent systems.

TABLE 7
Crystallization Conditions and Data

| Crystallization | Crystallization Conditions (mg of | Material after | |
|---|---|---|---|
| Solvent | TETN-2LA in mg solvent) | Crystallization | Micrographs |
| H₂O | 73 mg in 0.5 mL H₂O | Light peach glass | Crystals |
| | (1.5 weeks slow evaporation) | solid | within glass |
| H₂O | 382 mg in 1.0 mL H₂O | Light peach glass | Crystalline |
| | (slow evaporation) | solid | |
| Acetone-Hexane | 50 mg in 3 ml acetone:hexane (1 ml:2 ml), | Light peach glass | Crystalline + |
| | respectively | solid | Glass |
| MeOH | Slow evaporation | Oil | NA |
| IPA | Slow evaporation | Oil | NA |
| EtOH-Hexane | 78 mg in 3 ml EtOH:Hexane (1 ml:2 ml), | Gummy-oil | NA |
| | respectively | | |
| EtOH | 20 mg in 0.5 mL heated to dissolve | Oil | NA |
| | and slow evaporation | | |

Tetrandrine bis-pyruvate was formed by slowly adding a solution of pyruvic acid (14.118 g, 160.328 mmol) to a stirred solution of Tetrandrine (49.922 g, 80.164 mmol) in dichloromethane (DCM) (200 mL). The residual pyruvic acid was rinsed with dichloromethane (DCM) (25 mL) and added to the Tetrandrine solution. The mixture was stirred for 1 h and then concentrated to afford a solid foam. The foam was placed on the high vacuum overnight. The solid was then slurried with diethyl ether (200 mL) at room temperature for 20 minutes and filtered. The solid was placed onto the high vacuum over the weekend. The solid was then dissolved in ethanol (EtOH) (200 mL) and concentrated. The semi-solid foam was triturated with diethyl ether (100 mL) and concentrated. The solid was then placed onto high vacuum overnight to afford Tetrandrine bis-pyruvate (TETN bis-pyruvate) (60.15 g, 94%).

Tetrandrine bis-citrate was formed in an exemplary method as follows. To a stirred solution of Tetrandrine (3.0 g, 4.817 mmol) in ethanol-dichloromethane (EtOH-DCM) (100 mL/20 mL, respectively) was added solid citric acid (1.851 g, 9.634 mmol). The slurry was stirred at room temperature for 5 minutes and then water (10 mL) was added. The homogenous solution was stirred for 1 hour and was then concentrated to give a viscous oil. The oil was triturated with methyl tert butyl ether (1000 mL) resulting in a solid. Next, 180 mg of the solid was dissolved in hot 1:1 EtOH:Acetone (4 mL:4 mL, respectively) and allowed to cool with stirring. The resulting precipitate was centrifuged and the solvent decanted. A micrograph was obtained of the solid. Powder X-ray diffraction (PXRD) indicated amorphous material.

Accordingly, the present disclosure is directed at supplying of the alkaloids Cepharanthine or Tetrandrine, where such alkaloids include tertiary amine groups suitable for salt formation, followed by treatment of said tertiary amine groups with a selected inorganic or organic acid, and forming a pharmaceutically accepted salt thereof. The formation of the pharmaceutically acceptable salt also includes the use of a salt formation protocol that allows for isolation of the salt in a desired polymorphic form which provides enhanced water solubility. Such enhanced water solubility may then be relied upon to provide for much more efficient dosage regimes for treatment of viral infections, via routes of administration including, but not limited to oral administration and/or IV, IP or IM administration techniques.

What is claimed is:

1. A chlorine salt form of Cepharanthine comprising Cepharanthine 2HCl, wherein said salt indicates the following general structure: 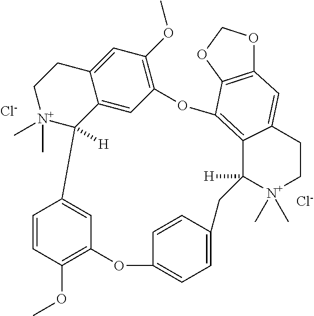 [SMILES: [Cl-] ; COc1ccc2cc1Oc1ccc(cc1)C[C@H]1c3c(cc4c(c3Oc3cc5c(cc3OC)CC[N+](C)(C)[C@H]25)OCO4)CC[N+]1(C)C ; [Cl-]]
wherein said salt indicates a plurality of distinguishing x-ray diffraction peaks at 2 Theta angles of 10-25 degrees as compared to non-distinguishing x-ray diffraction peaks at 2 Theta angles of greater than 25 degrees, wherein said distinguishing x-ray diffraction peaks have relative intensity counts between 200-550 at 2 Theta angles of 10-25 degrees and non-distinguishing x-ray diffraction peaks have relative intensity counts of less than 100 at 2 Theta angles of greater than 35 degrees.

2. A composition comprising the Cepharanthine 2HCl salt of claim 1 dispersed in a pharmaceutically acceptable carrier.

3. A chlorine salt form of Cepharanthine comprising Cepharanthine 2HCl, wherein said salt indicates the following general structure: 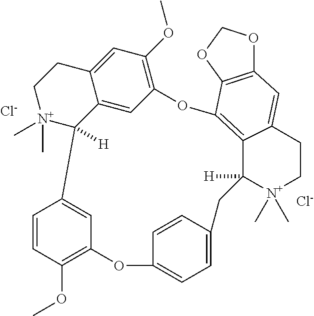 [SMILES: [Cl-] ; COc1ccc2cc1Oc1ccc(cc1)C[C@H]1c3c(cc4c(c3Oc3cc5c(cc3OC)CC[N+](C)(C)[C@H]25)OCO4)CC[N+]1(C)C ; [Cl-]]
wherein said salt indicates a plurality of distinguishing x-ray diffraction peaks at 2 Theta angles of 10-30 degrees as compared to non-distinguishing x-ray diffraction peaks at 2 Theta angles of greater than 30 degrees, wherein said distinguishing x-ray diffraction peaks have relative intensity counts between 150-500 at 2 Theta angles of 10-30 degrees and non-distinguishing x-ray diffraction peaks have relative intensity counts of less than 150 at 2 Theta angles of greater than 30 degrees.

4. A composition comprising the Cepharanthine 2HCl salt of claim 1 dispersed in a pharmaceutically acceptable carrier.

5. A chlorine salt form of Cepharanthine comprising Cepharanthine 2HCl, wherein said salt indicates the following general structure: 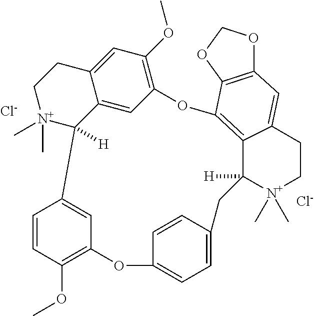 [SMILES: [Cl-] ; COc1ccc2cc1Oc1ccc(cc1)C[C@H]1c3c(cc4c(c3Oc3cc5c(cc3OC)CC[N+](C)(C)[C@H]25)OCO4)CC[N+]1(C)C ; [Cl-]]
a plurality of distinguishing x-ray diffraction peaks at 2 Theta angles of 10-25 degrees as compared to non-distinguishing x-ray diffraction peaks at 2 Theta angles of greater than 25 degrees, wherein said distinguishing x-ray diffraction peaks have relative intensity counts between 500-2500 at 2 Theta angles of 10-25 degrees and non-distinguishing x-ray diffraction peaks have relative intensity counts of less than 500 at 2 Theta angles of greater than 25 degrees.

6. A composition comprising the Cepharanthine 2HCl salt of claim 5 dispersed in a pharmaceutically acceptable carrier.
